(19) United States
(12) Patent Application Publication
(10) Pub. No.: US 2004/0072805 A1
(43) Pub. Date: Apr. 15, 2004

(54) Use of tumor necrosis factor inhibitors to treat cardiovascular disease

(76) Inventors: Marshelle S. Warren, Issaquah, WA (US); Theresa A. Deisher, Seattle, WA (US)

Correspondence Address: IMMUNEX CORPORATION, LAW DEPARTMENT, 1201 AMGEN COURT WEST, SEATTLE, WA, 98119, US

(21) Appl. No.: 10/115,625

(22) Filed: Apr. 3, 2002

Related U.S. Application Data
(60) Provisional application No. 60/282,244, filed on Apr. 6, 2001.

(51) Int. Cl. A61K 31/56

(52) U.S. Cl. 514/169

(57) ABSTRACT

The invention provides methods of identifying chronic heart failure patients who are likely to benefit from treatment with a TNFα inhibitor.

### CROSS REFERENCE TO RELATED APPLICATIONS

This application claims the benefit of priority under 35 U.S.C. §119 to U.S. Provisional Application Serial No. 60/282,244, filed Apr. 6, 2001, the disclosure of which is incorporated herein by reference.

### BACKGROUND OF THE INVENTION

Chronic heart failure (CHF), also called “congestive heart failure,” occurs when the heart is damaged from diseases such as high blood pressure, a heart attack, poor blood supply to the heart, a defective heart valve, atherosclerosis, rheumatic fever, heart muscle disease and so on. The failing heart becomes inefficient, resulting in fluid retention and shortness of breath, fatigue and exercise intolerance. CHF is defined by the symptom complex of dyspnea, fatigue and, in some patients, depressed left ventricular systolic function (ejection fraction <35-40%), and is an ultimate endpoint of all forms of serious heart disease.

Treatment of CHF has been directed primarily to prolonging the patient's life, although the benefits from treatment generally are assessed through improvement in other areas. For example, a reduced degree of dyspnea or improvement in performance in a standardized walking test have a substantial positive impact on the lifestyle of patients who live with this disease. An increased ejection fraction, which can be measured by echocardiogram or by multigated radionuclide ventriculography (MUGA), is another indicator of a successful treatment regimen.

It has been proposed that the pleiotropic cytokine TNFα may contribute to the progression of heart failure by exerting direct toxic effects on the heart and the circulation (see, e.g., Yokoyama et al., 1993; Torre-Amione et al., 1995). TNFα is a pleiotropic cytokine that is produced by the heart under certain forms of stress (Kapadia et al., 1995b; Kapadia et al., 1997). For example, patients with various types of heart disease have elevated levels of circulating TNFα, and the levels of TNFα have been shown to increase with disease progression (see, e.g., Maury et al., 1989; Levine et al., 1990; McMurrayetal., 1991; Han et al., 1992; Matsumori et al., 1994b; Satoh et al., 1997; Seta et al., 1996; Torre-Amione et al., 1996b).

Plasma cytokine parameters in patients with chronic heart failure have been reported in Rauchhaus et al., Circulation 102:3060-3067 (2000)). Other studies have addressed levels of plasma brain natriuretic peptide and interleukin-6 (IL-6) in chronic heart failure patients (Maeda et al., J Am College Cardiol 36(5): 1587-93).

Pathophysiologically relevant peripheral and/or elevated intramyocardial levels of TNFα are sufficient to mimic many aspects of the heart failure phenotype, including left ventricular dilation, left ventricular dysfunction, as well as activation of the fetal gene program (Suffredini et al., 1989; Hegewisch et al., 1990), hence it has been suggested that TNFα plays a contributory role in the pathogenesis of heart failure (see, e.g., Seta et al., 1996).

It has been suggested that suppression of TNFα might benefit CHF patients (e.g., McMurray et al., 1991), and many studies have provided support for this proposal. For example, TNFα has been shown in isolated hamster heart to inhibit contractility (Finkel et al., 1992). In mice, antibodies against TNFα were effective in ameliorating the severity of artificially-induced heart disease (Smith et al., 1992). In another study, TNFα-induced depression in left ventricle function in rats was partially reversed by administering the TNFα antagonist TNFR:Fc (Bozkurt et al., 1998), and in yet a different study, TNFR:Fc was shown to suppress the negative inotropic effect of TNF in cultured myocytes (Kapadia et al., 1995a). Others demonstrated that TNFR:Fc could reduce burn-induced myocardial dysfunction in guinea pigs (Giroir et al., 1994). Another study showed that vesnarinone, an agent used to treat CHF, could suppress lipoprotein-induced TNFα production human blood cells in vitro (Matsumori et al., 1994a). One group has proposed using TNFα antagonist to treat cardiovascular disorders related to thrombotic events, while another has proposed using adenosine as a means of reducing TNFα production in failing myocardial tissues (WO 97/30088; U.S. Pat. No. 5,998,386). In mice genetically modified to overexpress TNFα, it has been shown that myocardial extracellular matrix remodeling can be modulated by anti-TNFα therapy (Li et al., Proc Natl Acad Sci USA:97(23):12746-51). In other studies, it was shown that anti-TNFα therapy abrogated myocardial inflammation but not hypertrophy in mice that were overexpressing TNFα (Kubota et al., Circulation 101:2518-25 (2000)).

In one study, a small group of human CHF patients were given a single dose of TNFR:Fc, and fourteen days later exhibited decreased levels of circulating TNFα, increased ability to exercise, and improved symptomology (Deswal et al., 1997). In addition, the TNFα suppressor pentoxifylline reportedly induces improved left ventricle function concomitant with decreased levels of serum TNFα levels in patients with idiopathic dilated cardiomyopathy (Skudicky et al., 1998; Sliwa et al., 1998). The treatment of various heart diseases with TNFα antagonists is disclosed also in the following: U.S. Pat. No. 5,594,106; U.S. Pat. No. 5,629,285; U.S. Pat. No. 5,691,382; U.S. Pat. No. 5,700,838; U.S. Pat. No. 5,886,010; WO 91/15451; WO 94/10990; WO 95/19957; WO 96/21447; EP 0 453 898 B1; EP 0 486 809 A2; EP 0626389 A1).

TNFα binds to cells through two membrane receptor molecules having molecular weights of approximately 55 kDa and 75 kDa p55 and p75). In addition to binding TNFα, these same receptors mediate the binding to cells of TNFP, which is another cytokine associated with inflammation. TNFβ, also known as lymphotoxin-α (LTα), shares structural similarities with TNFα (Cosman, Blood Cell Biochem 7:51-77, 1996).

Although progress has been made in devising effective treatment for CHF in human patients, it is not expected that such treatment will be universally effective, and better methods are needed for determining prior to treatment which patients will respond, as well as better methods for determining which patients are responding during the treatment.

### SUMMARY OF THE INVENTION

The invention provides methods for identifying patients who qualify for the treatment of chronic heart failure and other cardiovascular disorders by administration of inhibitors of TNFα. Provided herein is a method of identifying a chronic heart failure patient who qualifies to receive treatment with a TNFα inhibitor. This method involves the level in the patient's serum of at least one substance used as a marker for the severity of heart disease. For this method, the substance measured is one of the following: TNFα; an interleukin associated with inflammation, including IL-1, IL-1 beta, IL-1 alpha, IL6, IL8, IL18; MMPs; TNF receptors (type I or II); serum creatinine; high sensitivity C-reactive protein (CRP) levels; troponin; BNP; cerimide; a chemokine; MCP-1; lymphotoxin α; an endothelin; an endothelin receptor; big endothelin; endothelin-2; endothelin receptor type A; endothelin receptor type B; an indicator of the andrenergic system; norepinephrine; epinephrine; an alpha andrenergic receptor; beta 1 andrenergic receptor; beta 2 andrenergic receptor; beta 3 andrenergic receptor; an indicator of the renin-angiotensin system; renin; angiotensin; aldosterone; an angiotensin converting enzymes; a natriuretic peptide family member; BNP; ANP; CNP; an acute phase protein; CRP; PIIINP; a nitric oxide synthase; INOS; an epithelial growth factor receptor; and an epithelial growth factor ligand. More than one of the above substances may be measured for this method.

If these measurements indicate that the patient's serum level of the measured substance is at least two times higher than the level of the same substance in the serum of a patient who does not have chronic heart failure, then it is thereby determined that the patient qualifies for treatment of their chronic heart failure by being administered a TNFα inhibitor.

In one embodiment of the invention, the TNFα inhibitor is TNFR:Fc, and the TNFR:Fc is administered to the patient by subcutaneous injection at a dose of 5 mg/m² or 12 mg/m² per dose up to a maximum of 25 mg per dose at least one time per week or two times per week for a time sufficient to induce a decrease over baseline of one or more of the substances measured in the patient's serum. Alternatively, a dose of 50 mg per dose is administered at least one time per week.

### DETAILED DESCRIPTION OF THE INVENTION

It is generally accepted in treating chronic heart failure (CHF) that not all patients will respond favorably to treatment. Provided herein are improved methods for treating chronic heart failure (CHF) and related conditions by administering an agent capable of reducing the level of TNFα or of blocking the interaction of TNFα and its receptors. As used herein, the terms “TNFα inhibitor” or “TNFα antagonist” includes agents that either reduce the effective amount of biologically active TNFα or block the synthesis or processing of the TNFα polypeptide. By using the methods provided herein, one can identify prior to initiating treatment which CHF patients are likely to respond to treatment with such agents.

As used herein, the term “CHF” encompasses related conditions that may lead to chronic heart failure, including but not limited to acute ischemic syndrome/unstable angina and atherosclerosis, and also includes conditions that may result from CHF, including poor blood supply to the heart; cardiomyopathy; (damaged heart muscle from any type of insult resulting in loss of viable myocardial tissue) and ventricular arrythmias. As used herein, “CHF” also includes patients with diastolic dysfunction, that is, patients who present with signs and symptoms of CHF but whose left ventricular ejection fraction measurements indicate that they do not have systolic dysfunction.

If a patient is predicted to respond to treatment with a TNFU inhibitor, the patient is referred to as “qualified” to receive treatment in accord with the invention.

In one embodiment of the invention, the patient qualifies for treatment if the patient presents with evidence of cardiac remodeling. Evidence of cardiac remodeling includes increased left ventricular end diastolic volume (LVEDV) as measured by echo cardiography or cardiac MRI. In people with normal hearts, an LVEDV has a mean value of 66+/−12 (ml/m²). CHF patients with cardiac remodeling may have an LVEDV of 1.5 to 3 times greater than normal. Another means of detecting cardiac remodeling is to measure the left ventricular end systolic volume LVESV, which in people with normal hearts has a mean value of 22+/−5 (ml/m²). CHF patients who qualify for treatment have an LVESV that is 2 to 8 times greater than. Other criteria for cardiac remodeling is left ventricular mass (LVM) (g/m²). In normals, the mean value is 87+/−12 g/m², and in patients who qualify for treatment in accordance with the subject methods, the LVM is 1.25 to 2 times greater than normal.

In another aspect of the invention, patients who qualify for treatment are identified by measuring their serum levels of procollagen type III amino-terminal peptide (PIIINP). Patients who qualify are characterized by having a serum level of PIITNP of >3.85 μg/l, a value for PIIINP that correlates with a poor outcome in CHF patients (Zannad et al., Circulation 102:2700-2706 (2000)).

In yet another embodiment of the invention, patients who qualify for treatment are identified by measuring their serum levels of matrix imetalloproteases (MMPs). Patients who qualify for treatment in accord with the invention have elevated MMP levels.

In another aspect of the invention, patients who qualify for treatment in accord with the subject methods are those who suffer from cardiac inflammation. Such patients can be identified by measuring serum levels of molecules associated with cardiac inflammation, including TNFα, IL-1 (including IL-1 beta), IL-6, IL-10, TNF receptors, serum creatinine, high sensitivity C-reactive protein (CRP) levels, troponin, BNP, cerimide. Patients who qualify will exhibit one of the following: serum TNFα greater than or equal to 2 pg/ml; serum IL-6 greater than or equal to 3 pg/ml; serum IL-10 levels that are less than normal; serum TNF receptor type I of equal to or greater than 1300 pg/ml, or serum TNF receptor type II levels of equal to or greater than 2000 pg/ml; serum creatinine between about 100-140 μm/l; high sensitivity C-reactive protein (CRP) levels greater than or equal to 0.3 mg/dl; troponin; brain natriuretic peptide (BNP) greater than or equal to 170 pg/ml.

The presence of certain TNFR polymorphisms also may be used as a means for identifying which patients will respond to treatment with TNFα inhibitors. Patients who qualify may have a p75 TNFR in which arginine is changed to proline at amino acid 143. In another embodiment, the patients who qualify are those having a p75 TNFR in which methionine is changed to arginine at amino acid 198. In another embodiment, the patients who qualify have a p75 TNFR in which alanine is changed to threonine at amino acid 365.

In another embodiment, patients who qualify for treatment are characterized by cachexia. Cachexia may be characterized by any convenient means. One means of determining that the patient exhibits cachexia is a finding that their body mass index (BMI) equal to or less than 24, a standard measure that is based on patient's height and weight.

In another aspect of the invention, patients who qualify for treatment are those who present with a New York Heart Association (NYHA) functional classification of Class I. Sufficiency of treatment is reached when the patient improves to the point where they no longer appear to suffer from CHF according to the NYHA criteria. These criteria are derived from the Committee of the New York Heart Association: Nomenclature and Criteria for Diagnosis of the Heart and Great Vessels (8^{th} Edition, Boston: Little, Brown and Co., 1979).

In another aspect of the invention, patients who qualify for treatment are those who present with a New York Heart Association (NYHA) functional classification of Class II. Sufficiency of treatment is reached when the patient improves to the point where he or she now is classified as NYHA Class I instead of Class II.

Classification according to the New York Heart Association (NYHA) criteria is performed as follows:

Class I

No limitation of physical activity. Ordinary physical activity does not cause undue fatigue, palpitation, or dyspnea.

Class II

Slight limitation of physical activity. Comfortable at rest, but ordinary physical activity results in fatigue, palpitation, or dyspnea.

Alternatively, one can identify patients who qualify for treatment based on detecting a serum level that is at least two times higher than the level found in patients having normal hearts of one or more of the following: TNFα; an interleukin associated with inflammation, including IL-1 (and particularly IL-1 beta), IL-6, IL-8, IL-18; MMPs; TNF receptors (type I or II); serum creatinine; high sensitivity C-reactive protein (CRP) levels; troponin; BNP; cerimide; chemokine family members, such as MCP-1; lymphotoxin α; endothelins and their receptors (big endothelin, endothelin-2, endothelin receptor types A or B; an indicator of the adrenergic system (norepinephrine, epinephrine, alpha adrenergic receptors, beta adrenergic receptors (beta 1, beta 2, or beta 3)); indicators of the renin-angiotensin system, including renin, angiotensin, aldosterone, angiotensin receptors (AT1 and AT2) and angiotensin converting enzymes; natriuretic peptide family members, including BNP, ANP or CNP; acute phase proteins, including CRP; PIIINP; nitric oxide synthases, including INOS; and epithelial growth factor receptors and ligands. In addition, patients qualify may be identified by having an at least a two-fold decrease as compared with normals of their serum level of IL-10.

In one embodiment of the invention, a CHF patient is administered a TNFα antagonist that is capable of inhibiting the binding of TNFα to a TNFα receptor. In a preferred embodiment of the invention, the TNFα antagonist is a soluble TNF receptor comprising all or part of the extracellular region of the p55 or the p75 TNF receptor. In a particularly preferred embodiment, the antagonist is one that mimics the 75 kDa TNFR and that binds to TNFα in the patient's body. Once bound to the antagonist, the TNFα is prevented from binding its natural receptor, and thus cannot manifest its biological activities. A TNFα antagonist suitable for use in these methods is recombinant TNFR:Fc (hereafter referred to as “TNFR:Fc” or “etanercept”). Etanercept is currently sold by Immunex Corporation under the trade name ENBRIEL,® and is a dimer of two molecules of the extracellular portion of the p75 TNFα receptor, each molecule consisting of a 235 amino acid polypeptide that is fused to a 232 amino acid Fc portion of human IgG₁. In addition to etanercept, the use of other soluble mimics of the p75 molecule for treating CHF are within the scope of the invention. Etanercept is a dimeric TNFR that competes for TNFα with the receptors on the cell surface, thus inhibiting TNFα from binding to the cell. In contrast to many other types of TNF inhibitor, inhibitors comprising a TNFR are capable also of binding to the inflammatory cytokine LTα. Thus, TNFR:Fc has the capacity to suppress the binding of LTα to its natural receptors, which may contribute to the potency of TNFR:Fc.

The subject TNFα inhibitors are capable of reducing the effective amount of endogenous biologically active TNFα, such as by reducing the amount of TNFα produced, or by preventing the binding of TNFα to its cell surface receptor (TNFR). Agents capable of reducing production of TNFα include, for example, adenosine, which may be administered as described in U.S. Pat. No. 5,998,386, and antisense oligonucleotides or ribozymes that inhibit TNFα production. Other antagonists useful for inhibiting the binding of TNFα and TNFR include receptor-binding peptide fragments of TNFα, antibodies directed against TNFα, and recombinant proteins comprising all or portions of receptors for TNFα or modified variants thereof, including genetically-modified muteins, multimeric forms and sustained-release formulations. In other embodiments of the invention, the diseases discussed herein are treated with molecules that inhibit the formation of the IgA-α₁AT complex, such as the peptides disclosed in EP 0 614 464 B, or antibodies against this complex. The hereindescribed conditions also may be treated with disaccharides, sulfated derivatives of glucosamine or other similar carbohydrates as described in U.S. Pat. No. 6,020,323. In addition, the hereindescribed diseases may be treated with the peptide TNFα inhibitors disclosed in U.S. Pat. No. 5,641,751 and U.S. Pat. No. 5,519,000, and the D-amino acid-containing peptides described in U.S. Pat. No. 5,753,628. In addition, the conditions described herein may be treated with inhibitors of TNTα converting enzyme.

Any TNFα inhibitor that is a protein may be delivered to the patient by viral vector (such as an adenovirus or a retrovirus) that expresses the proteinaceous TNFα inhibitor.

Other compounds suitable for treating the cardiovascular diseases described herein include small molecules such as thalidomide or thalidomide analogs, pentoxifylline, or matrix metalloproteinase (MMP) inhibitors or other small molecules. Suitable MMP inhibitors include, for example, those described in U.S. Pat. Nos. 5,883,131, 5,863,949 and 5,861,510 as well as the mercapto alkyl peptidyl compounds described in U.S. Pat. No. 5,872,146. Other small molecules capable of reducing TNFα production, include, for example, the molecules described in U.S. Pat. Nos. 5,508,300, 5,596,013 and 5,563,143, any of which can be administered in combination with TNFα inhibitors such as soluble TNFRs or antibodies against TNFα. Additional small molecules useful for treating the TNFα-mediated diseases described herein include the MMP inhibitors that are described in U.S. Pat. No. 5,747,514, U.S. Pat. No. 5,691,382, as well as the hydroxamic acid derivatives described in U.S. Pat. No. 5,821,262. The diseases described herein also may be treated with small molecules that inhibit phosphodiesterase IV and TNFα production, such as substituted oxime derivatives (WO 96/00215), quinoline sulfonamides (U.S. Pat. No. 5,834,485), aryl furan derivatives (WO 99/18095) and heterobicyclic derivatives (WO 96/01825; GB 2 291 422 A). Also useful are thiazole derivatives that suppress TNFα and IFNγ (WO 99/15524), as well as xanthine derivatives that suppress TNFct and other proinflammatory cytokines (see, for example, U.S. Pat. No. 5,118,500, U.S. Pat. No. 5,096,906 and U.S. Pat. No. 5,196,430). Additional small molecules useful for treating the hereindescribed conditions include those disclosed in U.S. Pat. No. 5,547,979.

Also included among the TNFα inhibitors of the invention are antisense oligonucleotides that act to directly block the translation of mRNA by hybridizing to targeted mRNA and preventing polypeptide translation. Antisense oligonucleotides are suitable for use in treating any of the medical disorders disclosed herein, either alone or in combination with other TNFα inhibitors, such as TNMR:Fc, or in combination with other agents for treating the same condition. Antisense molecules of the invention may interfere with the translation of TNFα, a TNFα receptor, or an enzyme in the metabolic pathways for the synthesis of TNFα. Absolute complementarity, although preferred, is not required. A sequence “complementary” to a portion of a nucleic acid, as referred to herein, means a sequence having sufficient complementarity to be able to hybridize with the nucleic acid, forming a stable duplex (or triplex, as appropriate). The ability to hybridize will depend on both the degree of complementarity and the length of the antisense nucleic acid. Oligonucleotides that are complementary to the 5′ end of the message, e.g., the 5′ untranslated sequence up to and including the AUG initiation codon, should work most efficiently at inhibiting translation. However, oligonucleotides complementary to either the 5′- or 3′-non-translated, non-coding regions of the targeted transcript can be used. Oligonucleotides complementary to the 5′ untranslated region of the mRNA should include the complement of the AUG start codon. Antisense nucleic acids will be at least six nucleotides in length, or 6-50 nucleotides in length, and preferably will contain 18-21 nucleotides. Chemically modified oligonucleotides may be used, such as those described in U.S. Pat. No. 6,114,517, which describes the use for this purpose of phosphorothioates, phosphorodithioates, phosphotriesters, aminoalkylphosphotriesters, methyl and other alkyl phosphonates, various phosphonates, phosphinates, and phosphoramidates and so on. Antisense oligonucleotides for suitable for treating diseases associated with elevated TNFα include, for example, the anti-TNFα oligonucleotides described in U.S. Pat. No. 6,080,580, which proposes the use of such oligonucleotides as candidates for testing in animal models of diabetes mellitus, rheumatoid arthritis, contact sensitivity, Crohn's disease, multiple sclerosis, pancreatitis, hepatitis and heart transplant. Antisense oligonucleotides can be administered parenterally, including by intravenous or subcutaneous injection, or they can be incorporated into formulations suitable for oral administration, such as, for example, ISIS 104838, which targets TNFα.

Ribozyme molecules designed to catalytically cleave mRNA transcripts can also be used to prevent the translation of mRNAs encoding TNFα, TNFα receptors, or enzymes involved in synthesis of TNFα or TNFRs (see, e.g., PCT WO90/11364; U.S. Pat. No. 5,824,519). Ribozymes useful for this purpose include hammerhead ribozymes (Haseloff and Gerlach, 1988, Nature, 334:585-591), RNA endoribonucleases (hereinafter “Cech-type ribozymes”) such as the one that occurs naturally in Tetrahymena thermophila (known as the IVS, or L-19 IVS RNA) (see, for example, WO 88/04300; Been and Cech, 1986, Cell, 47:207-216). Ribozymes can be composed of modified oligonucleotides (e.g. for improved stability, targeting, etc.) and should be delivered to cells which express the target peptide in vivo. A preferred method of delivery involves using a DNA construct encoding the ribozyme under the control of a strong constitutive pol III or pol II promoter, so that transfected cells will produce sufficient quantities of the ribozyme to destroy endogenous target mRNA, thereby inhibiting its translation.

Alternatively, expression of genes involved in TNFα or TNFR production can be reduced by targeting deoxyribonucleotide sequences complementary to the regulatory region of the target gene (i.e., the target gene promoter and/or enhancers) to form triple helical structures that prevent transcription of the target gene. (see, for example, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Soluble forms of TNFRs useful as antagonists for the subject methods may include monomers, fusion proteins (also called “chimeric proteins), dimers, trimers or higher order multimers. The soluble TNFR mimics of the present invention may be derived from TNFRs p55 or p75 or fragments thereof. TNFRs other than p55 and p75 also are useful for deriving soluble compounds for treating the various medical disorders described herein, such for example the TNFR that is described in WO 99/04001. Soluble TNFR molecules used to construct TNFR mimics include, for example, analogs or fragments of native TNFRs having at least 20 amino acids, that lack the transmembrane region of the native TNFR, and that are capable of binding TNFα. Antagonists derived from TNFRs compete for TNFα with the receptors on the cell surface, thus inhibiting TNFα from binding to cells, thereby preventing it from manifesting its biological activities. Binding of soluble TNFRs to TNFα or LTα can be assayed using ELISA or any other convenient assay. This invention provides for the use of soluble TNFα receptors in the manufacture of medicaments for the treatment of numerous diseases.

The soluble TNFR polypeptides or fragments of the invention may be fused with a second polypeptide to form a chimeric protein. The second polypeptide may promote the spontaneous formation by the chimeric protein of a dimer, trimer or higher order number that is capable of binding a TNFα or a LTα molecule and preventing it from binding to cell-bound receptors. Chimeric proteins used as antagonists include, for example, molecules derived from the constant region of an antibody molecule and the extracellular portion of a TNFR. Such molecules are referred to herein as TNFR-Ig fusion proteins. A preferred TNFR-Ig fusion protein suitable for treating diseases in humans and other mammals is recombinant TNFR:Fc, a term which as used herein refers to “etanercept,” which is a dimer of two molecules of the extracellular portion of the p75 TNFα receptor, each molecule consisting of a 235 amino acid TNFR-derived polypeptide that is fused to a 232 amino acid Fc portion of human IgG₁. Etanercept is currently sold by Immunex Corporation under the trade name ENBREL.® Because the p75 receptor protein that it incorporates binds not only to TNFα, but also to the inflammatory cytokine LTα, etanercept can act as a competitive inhibitor not only of TNFα, but also of LTD. This is in contrast to antibodies directed against TNFα, which cannot inhibit LTα. Also encompassed by the invention are treatments using a compound that comprises the extracellular portion of the 55 kDa TNFR fused to the Fc portion of IgG, as well as compositions and combinations containing such a molecule. Encompassed also are therapeutic methods involving the administration of soluble TNFRs derived from the extracellular regions of TNFα receptor molecules other than the p55 and p75 TNFRs, such as for example the TNFR described in WO 99/04001, including TNFR-Ig's derived from this TNFR. Other suitable TNFα inhibitors include the humanized anti-TNFα antibody D2E7 (Knoll Pharmaceutical/BASF AG).

In one preferred embodiment of the invention, sustained-release forms of soluble TNFRs are used, including sustained-release forms of TNFR:Fc. Sustained-release forms suitable for use in the disclosed methods include, but are not limited to, TNFRs that are encapsulated in a slowly-dissolving biocompatible polymer (such as the alginate microparticles described in U.S. Pat. No. 6,036,978 or the polyethylene-vinyl acetate and poly(lactic-glucolic acid) compositions described in U.S. Pat. No. 6,083,534), admixed with such a polymer (including topically applied hydrogels), and or encased in a biocompatible semi-permeable implant. In addition, a soluble TNFR type I or type II for use in the hereindescribed therapies may be conjugated with polyethylene glycol (pegylated) to prolong its serum half-life or to enhance protein delivery.

In accord with this invention, patients identified as qualified to receive treatment are administered a therapeutically effective amount of a TNFα inhibitor. In one preferred embodiment, the TNFα inhibitor is a soluble TNFR. Most preferably, the soluble TNFR is TNFR:Fc. As used herein, the phrase “administering a therapeutically effective amount” of a therapeutic agent means that the patient is treated with the agent in an amount and for a time sufficient to induce an improvement in the chosen indicator or indicators as described above.

Any efficacious route of administration may be used to therapeutically administer TNFR:Fc or other TNFα antagonist. If injected, the TNFα inhibitor can be administered, for example, via intra-articular, intravenous, intramuscular, intralesional, intraperitoneal or subcutaneous routes by bolus injection or by continuous infusion. Other suitable means of administration include sustained release from implants, aerosol inhalation, eyedrops, oral preparations, including pills, syrups, lozenges or chewing gum, and topical preparations such as lotions, gels, sprays, ointmrents or other suitable techniques. Alternatively, proteinaceous TNFα inhibitors, such as a soluble TNFR, may be administered by implanting cultured cells that express the protein, for example, by implanting cells that express TNFR:Fc. In one embodiment, the patient's own cells are induced to produce TNFR:Fc by transfection in vivo or ex vivo with a DNA that encodes TNFR:Fc. This DNA can be introduced into the patient's cells, for example, by injecting naked DNA or liposome-encapsulated DNA that encodes TNFR:Fc, by infection with a viral vector expressing the DNA, or by other means known in the art. When TNFR:Fc is administered in combination with one or more other biologically active compounds, these may be administered by the same or by different routes, and may be administered simultaneously, separately or sequentially.

TNF inhibitors according to the invention preferably are administered in the form of a physiologically acceptable composition comprising purified recombinant protein in conjunction with physiologically acceptable carriers, excipients or diluents. Such carriers are nontoxic to recipients at the dosages and concentrations employed. Ordinarily, the preparation of such compositions entails combining the TNFα antagonist with buffers, antioxidants such as ascorbic acid, low molecular weight polypeptides (such as those having fewer than 10 amino acids), proteins, amino acids, carbohydrates such as glucose, sucrose or dextrins, chelating agents such as EDTA, glutathione and other stabilizers and excipients. Neutral buffered saline or saline mixed with conspecific serum albumin are exemplary appropriate diluents, In accordance with appropriate industry standards, preservatives may also be added, such as benzyl alcohol. TNFR:Fc preferably is formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in Remington's Pharmaceutical Sciences, 16^{th} Ed., Mack Publishing Company, Easton, Pa., 1980.

Appropriate dosages can be determined in standard dosing trials, and may vary according to the chosen route of administration. The amount and frequency of administration will depend on such factors as the nature and severity of the indication being treated, the desired response, the age and condition of the patient, and so forth.

In one preferred embodiment of the invention, the TNFα inhibitor is TNFR:Fc which is administered one time per week to treat the various medical disorders disclosed herein, in another embodiment is administered at least two times per week, and in another embodiment is administered at least three times per week. An adult patient is a person who is 18 years of age or older. If injected, the effective amount of TNFR:Fc per adult dose ranges from 1-20 mg/m, and preferably is about 5-12 mg/m². Alternatively, a flat dose may be administered, whose amount may range from 5-100 mg/dose. Exemplary dose ranges for a flat dose to be administered by subcutaneous injection are 5-25 mg/dose, 25-50 mg/dose and 50-100 mg/dose. In one embodiment of the invention, the various indications described below are treated by administering a preparation acceptable for injection containing TNFR:Fc at 25 mg/dose, or alternatively, containing 50 mg per dose. The 25 mg or 50 mg dose may be administered repeatedly, particularly for chronic conditions. If a route of administration other than injection is used, the dose is appropriately adjusted in accord with standard medical practices. In many instances, an improvement in a patient's condition will be obtained by injecting a dose of about 25 mg of TNFR:Fc one to three times per week over a period of at least three weeks, or a dose of 50 mg of TNFR:Fc one or two times per week for at least three weeks, though treatment for longer periods may be necessary to induce the desired degree of improvement. For incurable chronic conditions, the regimen may be continued indefinitely, with adjustments being made to dose and frequency if such are deemed necessary by the patient's physician.

For pediatric patients (age 4-17), a suitable regimen involves the subcutaneous injection of 0.4 mg/kg, up to a maximum dose of 25 mg of TNFR:Fc, administered by subcutaneous injection one or more times per week.

Additionally, TNFR:Fc may be combined with a second TNFα antagonist, including an antibody against TNFα or TNFR, a TNFα-derived peptide that acts as a competitive inhibitor of TNFα (such as those described in U.S. Pat. No. 5,795,859 or U.S. Pat. No. 6,107,273), a TNFR-IgG fusion protein other than etanercept, such as one containing the extracellular portion of the p55 TNFα receptor, a soluble TNFR other than an IgG fusion protein, or other molecules that reduce endogenous TNFα levels, such as inhibitors of the TNFα converting enzyme (see e.g., U.S. Pat. No. 5,594,106), or any of the small molecules or TNFα inhibitors that are described above, including pentoxifylline or thalidomide.

If an antibody against TNFα is used as the TNFα inhibitor, a preferred dose range is 0.1 to 20 mg/kg, and more preferably is 1-10 mg/kg. Another preferred dose range for anti-TNFα antibody is 0.75 to 7.5 mg/kg of body weight. Humanized antibodies are preferred, that is, antibodies in which only the antigen-binding portion of the antibody molecule is derived from a non-human source. An exemplary humanized antibody for treating the hereindescribed diseases is infliximab (sold by Centocor as REMICADE®), which is a chimeric IgG1κ monoclonal antibody having an approximate molecular weight of 149,100 daltons. Infliximab is composed of human constant and murine variable regions, and binds specifically to human TNFα. Other suitable anti-TNFα antibodies include the humanized antibodies D2E7 and CDP571, and the antibodies described in EP 0 516 785 B1, U.S. Pat. No. 5,656,272, EP 0 492 448 A1. Such antibodies may be injected or administered intravenously.

The present invention also relates to the use of the disclosed TNFα inhibitors, such as TNFR:Fc, in the manufacture of a medicament for the prevention or therapeutic treatment of each medical disorder disclosed herein.

In one embodiment of the invention, CHF patients who are being treated with TNFR:Fc are treated concurrently with one or more of the following: a diuretic; an ACE inhibitor; digoxin; an angiotensin II antagonist; a beta blocker; amiodarone; a nitrate; and hydralazine. In yet another embodiment, patients being treated with a TNFα inhibitor, such as TNFR:Fc, are treated concurrently with one or more treatment selected from the following: an additional cytokine inhibitor, such as an IL-1 inhibitor; a neurohormonal antagonist (such as an ACE inhibitor, a beta blocker, an endothelin antagonist), left ventricular assist device (LVAD); or a biventricular pacing device.

An IL-1 inhibitor, such as soluble IL-1 type II receptor, may also be administered in addition to the TNFα inhibitor.

Various indicators that reflect the patient's degree of heart failure or other heart condition may be assessed for determining whether the amount and time of the treatment is sufficient. The baseline value for the chosen indicator or indicators is established by examination of the patient within about 60 days prior to administration of the first dose of the etanercept or other TNFα-binding molecule.

If TNFR:Fc is used as the TNFα inhibitor and it is administered by injection, the effective amount per dose will range from 1-20 mg/m², and preferably is about 5-12 mg/m². Alternatively, a flat dose may be administered, whose amount may range from 5-100 mg/dose. An exemplary range for a flat dose is about 20-30 mg per dose. In one embodiment of the invention, a flat dose of 25 mg/dose or 50 mg/dose is repeatedly administered by subcutaneous injection. If a route of administration other than injection is used, the dose is appropriately adjusted in accord with standard medical practices.

Regardless of route of administration, it should be understood that the specific dose level and frequency of administration for a given patient may depend upon a variety of factors such as their age, body weight, general health, sex, diet, time of administration, other drugs being concurrently administered, side-effects the patient may experience and the severity of their heart disease.

In one of the preferred embodiments of the invention, chronic heart failure is treated by administering to the patient by subcutaneous injection a dose of TNFR:Fc at 5 mg/m² or 12 mg/m² per dose up to a maximum of 25 mg per dose at least two times per week for a time sufficient to induce a 10%, or more preferably a 30% reduction in serum level of one or more of the above indicators that was found to be pathologically elevated within 60 days prior to the initiation of treatment. If IL-10 is the marker, treatment is sufficient when IL-10 levels have become elevated by at least 10%, or more preferably by at least 30%. Generally, treatment is expected to for last at least 2-4 weeks before an improvement is observed. However, treatment may be continued for 1-6 months, 1-12 months, or indefinitely. Long-term treatment may be administered at the original dose or at a reduced maintenance dose. Moreover, if the treatment is discontinued for any reason, the treatment may be resumed if the patient's condition should worsen or recur.

In addition to subcutaneous injection, any other efficacious route of administration may be used to therapeutically administer TNFR:Fc or other TNFα antagonist. TNFR:Fc can be administered to a CHF or other heart disorder patient, for example, via intra-articular injection, intramuscular injection, intraperitoneal infusion or bolus injection, continuous infusion into a vein or artery, intrathecal or subdural injection, sustained release from implants, aerosol inhalation, suppository, oral preparations, such as tablets, capsules, pills or syrups, transdermal patch, biodegradable microcapsules or other suitable techniques, such as in vivo or ex vivo transfection of the patient's cells with recombinant DNA expressing a TNFR:Fc polypeptide. In other embodiments, antisense oligonucleotides are used to suppress TNFα synthesis in the patient's cells, or the patient may be administered cells that express high levels of an endogenously-encoded or a recombinant soluble TNFα receptor.

Typically, TNFα inhibitors are administered in the form of a composition comprising purified recombinant protein in conjunction with physiologically acceptable carriers, excipients or diluents. Such carriers should be nontoxic to recipients at the dosages and concentrations employed. Ordinarily, the preparation of such compositions entails combining the TNFα inhibitor with buffers, antioxidants such as ascorbic acid, low molecular weight polypeptides (such as those having fewer than 10 amino acids), proteins, amino acids, carbohydrates such as glucose, sucrose or dextrins, chelating agents such as EDTA, glutathione and other stabilizers and excipients. Neutral buffered saline or saline mixed with conspecific serum albumin are exemplary appropriate diluents. Preferably, when TNFR:Fc is used, it is formulated as a lyophilizate using appropriate excipient solutions (e.g., sucrose) as diluents. Appropriate dosages can be determined in standard dosing trials, and may vary according to the route of administration that is chosen. In accordance with appropriate industry standards, preservatives may also be added, such as benzyl alcohol. The amount and frequency of administration will depend, of course, on such factors as the nature and severity of the indication being treated, the desired response, the condition of the patient, and so forth.

The compositions described herein preferably are administered at least one time per week. In a preferred embodiment of the invention, TNFR:Fc is administered at least two times per week, and in another preferred embodiment, it is administered at least three times a week.

Patients treated in accordance with the invention may also be receiving other therapy for heart failure including a diuretic with an ACE inhibitor, digoxin, angiotensin II antagonist, beta blocker, amiodarone, nitrates, or hydralazine.

What is claimed is:

1. A method of identifying a chronic heart failure patient who qualifies to receive treatment with a TNFα inhibitor, said method comprising:
measuring in the patient's serum the level of a marker comprising at least one substance selected from the group consisting of TNFA; an interleukin associated with inflammation, including IL-1, IL-1 beta, IL-1 alpha, IL6, IL8, IL18; MMPs; TNF receptors (type I or II); serum creatinine; high sensitivity C-reactive protein (CRP) levels; troponin; BNP; cerimide; a chemokine; MCP-1; lymphotoxin α; an endothelin; an endothelin receptor; big endothelin; endothelin-2; endothelin receptor type A; endothelin receptor type B; an indicator of the andrenergic system; norepinephrine; epinephrine; an alpha andrenergic receptor; beta 1 andrenergic receptor; beta 2 andrenergic receptor; beta 3 andrenergic receptor; an indicator of the renin-angiotensin system; renin; angiotensin; aldosterone; an angiotensin converting enzymes; a natriuretic peptide family member; BNP; ANP; CNP; an acute phase protein; CRP; PIIINP; a nitric oxide synthase; INOS; an epithelial growth factor receptor; an epithelial growth factor ligands; and
determining that the patient's serum level of said substance is at least two times higher than the level of the same substance in the serum of a patient who does not have chronic heart failure.

2. The method of claim 1, wherein the TNFα inhibitor is TNFR:Fc, and the TNFR:Fc is administered to the patient by subcutaneous injection at a dose of 5 mg/m² or 12 mg/m² per dose up to a maximum of 25 mg per dose at least two times per week for a time sufficient to induce an improvement over baseline of one or more of the markers set forth in claim 1.

3. The method of claim 1, wherein the TNFα inhibitor is TNFR:Fc, and the TNFR:Fc is administered to the patient by subcutaneous injection at a dose of 50 mg per week at least one time pere week for a time sufficient to induce an improvement over baseline of one or more of the markers set forth in claim 1.
